(19) United States
(12) Patent Application Publication
(10) Pub. No.: US 2026/0337264 A1
(43) Pub. Date: Oct. 29, 2026

(54) ANTI-RHD ANTIBODIES FOR TREATING INFLAMMATORY DERMAL CONDITION

(71) Applicant: Jamie Nicole Heitmeyer, Santa Monica, CA (US)

(72) Inventor: Jamie Nicole Heitmeyer, Santa Monica, CA (US)

(21) Appl. No.: 19/107,189

(22) PCT Filed: Aug. 31, 2023
(86) PCT No.: PCT/US2023/073211

§ 371 Date: Feb. 27, 2025

Related U.S. Application Data
(60) Provisional application No. 63/374,097, filed on Aug. 31, 2022.

(51) Int. Cl. C07K 16/28; A61K 9/00; A61K 39/00; A61P 17/00

(52) U.S. Cl. CPC C07K 16/28; A61K 9/0019; A61P 17/00; A61K 2039/505; A61K 2039/54

(57) ABSTRACT

Provided herein is a method of treating, alleviating, or preventing one or more symptoms of an inflammatory dermal condition with an anti-RhD antibody. Also provided herein is a method of treating, alleviating, or preventing one or more symptoms of an IgE-mediated mast cell allergic response with an anti-RhD antibody.

### CROSS REFERENCE TO RELATED APPLICATION

This application claims the benefit of the priority of U.S. Provisional Application No. 63/374,097, filed Aug. 31, 2022; the disclosure of which is incorporated herein by reference in its entirety.

### FIELD

Provided herein is a method of treating, alleviating, or preventing one or more symptoms of an inflammatory dermal condition with an anti-RhD antibody. Also provided herein is a method of treating, alleviating, or preventing one or more symptoms of an IgE-mediated mast cell allergic response with an anti-RhD antibody.

### BACKGROUND

The Rh blood group with variable expressions of a D antigen is one of the complex systems in immunohematology. Avent and Reid, Blood 2000, 95, 375-87; Bras et al., Tzu. Chi. Med. J. 2019, 32, 167-70. Human blood groups were divided into two major groups depending on the presence (RhD-positive) or absence (RhD-negative) of the D antigen. Bras et al., Tzu. Chi. Med. J. 2019, 32, 167-70. The D antigen is highly immunogenic. Dean. Blood Groups and Red Cell Antigens [Internet]. Bethesda (MD); National Center for Biotechnology Information (US); 2005. People who do not produce the D antigen will produce anti-RhD antibodies if they encounter the D antigen on transfused RBCs (causing a hemolytic transfusion reaction) or on fetal RBCs (causing hemolytic disease). Id. Mast cells are ubiquitously expressed in tissue cells that mediate end stages of various dermatological disease processes, including Type 1 hypersensitivity initiated by an IgE-antigen, urticaria, atopic dermatitis, and eczema. Conti et al., Int. J. Mol. Sci. 2021, 22, 8076; Numata et al., Frontiers in Immunol. 2022, 13, 923495. There is a need for an effective therapy for treating inflammatory dermal condition.

### SUMMARY OF THE DISCLOSURE

Provided herein is a method of treating, alleviating, or preventing one or more symptoms of an inflammatory dermal condition in a subject, comprising administering to the subject in need thereof a therapeutically effective amount of an anti-RhD antibody.

Also provided herein is a method of treating, alleviating, or preventing one or more symptoms of an IgE-mediated mast cell allergic response in a subject, comprising administering to the subject in need thereof a therapeutically effective amount of an anti-RhD antibody.

Additionally, provided herein is a method of treating, alleviating, or preventing one or more symptoms of psoriasis in a subject, comprising administering to the subject in need thereof a therapeutically effective amount of an anti-RhD antibody.

### ILLUSTRATIVE EMBODIMENTS

Embodiment 1. A method of treating symptoms of an IgE-mediated mast cell allergic response in a patient in need thereof, comprising the step of administering to said patient at least one injection of human anti-RhD antibodies diluted in an appropriate biological buffer in an amount sufficient to alleviate said symptoms of said IgE-mediated mast cell or peripheral blood basophil allergic response.

Embodiment 2. The method of embodiment 1, wherein said IgE-mediated mast cell allergic response is selected from the group consisting of allergies, Type I hypersensitivity, anaphylaxis, hay fever, asthma, arthritis, urticaria, atopic dermatitis, rashes, heart disease and multiple sclerosis.

Embodiment 3. A method of inhibiting, in a patient in need thereof, a mast cell allergic response, comprising the step of administering to said patient human anti-RhD antibodies in an amount sufficient to inhibit said IgE-mediated mast cell allergic response.

Embodiment 4. A method of preventing or treating atopic dermatitis in a patient in need thereof, comprising the step of administering to said patient human anti-RhD antibodies in an amount sufficient to prevent or decrease symptoms of the disease.

Embodiment 5. A method of preventing or treating urticaria in a patient in need thereof, comprising the step of administering to said patient human anti-RhD antibodies in an amount sufficient to prevent or decrease symptoms of the disease.

Embodiment 6. A method of preventing or treating eczema in a patient in need thereof, comprising the step of administering to said patient human anti-RhD antibodies in an amount sufficient to prevent or decrease symptoms of the disease.

Embodiment 8. A method of preventing or treating contact dermatitis in a patient in need thereof, comprising the step of administering to said patient human anti-RhD antibodies in an amount sufficient to prevent or decrease symptoms of the disease.

Embodiment 9. A method of preventing or treating pruritus in a patient in need thereof, comprising the step of administering to said patient human anti-RhD antibodies in an amount sufficient to prevent or decrease symptoms of the disease.

Embodiment 10. A method of preventing or treating psoriasis in a patient in need thereof, comprising the step of administering to said human anti-RhD antibodies in an amount sufficient to prevent or decrease symptoms.

Embodiment 11. A method of treating symptoms of an IgE-mediated mast cell allergic response in a patient in need thereof, comprising the step of administering to said patient at topical application with human anti-RhD antibodies in an amount (0.00001-100 μg/mL) in an appropriate vehicle sufficient to alleviate said symptoms of said IgE-mediated mast cell allergic response.

Embodiment 12. The method of embodiment 11, wherein said IgE-mediated mast cell allergic response is selected from the group consisting of allergies, Type I hypersensitivity, anaphylaxis, hay fever, asthma, arthritis, urticaria, atopic dermatitis, rashes, heart disease and multiple sclerosis.

Embodiment 13. The method of embodiment 11 in a patient in need thereof, a mast cell allergic response, comprising the step of administering to said patient human anti-RhD antibodies topically in an amount sufficient to inhibit said IgE-mediated mast cell allergic response.

Embodiment 14. The method of embodiment 11 of preventing or treating atopic dermatitis in a patient in need thereof, comprising the step of administering to said patient human anti-RhD antibodies topically in an amount sufficient to prevent or decrease symptoms of the disease.

Embodiment 15. The method of embodiment 11 of preventing or treating urticaria in a patient in need thereof, comprising the step of administering to said patient human anti-RhD antibodies topically in an amount sufficient to prevent or decrease symptoms of the disease.

Embodiment 16. The method of embodiment 11 of preventing or treating eczema in a patient in need thereof, comprising the step of administering to said patient human anti-RhD antibodies topically in an amount sufficient to prevent or decrease symptoms of the disease.

Embodiment 17. The method of embodiment 11 of preventing or treating contact dermatitis in a patient in need thereof, comprising the step of administering to said patient human anti-RhD antibodies topically in an amount sufficient to prevent or decrease symptoms of the disease.

Embodiment 18. The method of embodiment 11 of preventing or treating pruritis in a patient in need thereof, comprising the step of administering to said patient human anti-RhD antibodies topically in an amount sufficient to prevent or decrease symptoms of the disease.

Embodiment 19. The method of embodiment 11 of preventing or treating psoriasis in a patient in need thereof, comprising the step of administering to said patient human anti-RhD antibodies topically in an amount sufficient to prevent or decrease symptoms of the disease.

### DETAILED DESCRIPTION

To facilitate understanding of the disclosure set forth herein, a number of terms are defined below.

Generally, the nomenclature used herein and the laboratory procedures in biochemistry, biology, immunology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “therapeutically effective amount” is meant to include the amount of an anti-RhD antibody that, when administered, is sufficient to prevent development of, or alleviate to some extent, one or more of the symptoms of the disorder, disease, or condition being treated.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 2nd ed.; Gibson Ed.; CRC Press, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

### Methods of Use

In one embodiment, provided herein is a method of treating, alleviating, or preventing one or more symptoms of an IgE-mediated mast cell allergic response in a subject, comprising administering to the subject in need thereof a therapeutically effective amount of an anti-RhD antibody.

In certain embodiments, the IgE-mediated mast cell allergic response is inflammatory dermal condition. In certain embodiments, the IgE-mediated mast cell allergic response is an allergy, Type I hypersensitivity, anaphylaxis, hay fever, asthma, arthritis, urticaria, atopic dermatitis, rash, a heart disease, or multiple sclerosis. In certain embodiments, the IgE-mediated mast cell allergic response is an allergy. In certain embodiments, the IgE-mediated mast cell allergic response is Type I hypersensitivity. In certain embodiments, the IgE-mediated mast cell allergic response is anaphylaxis. In certain embodiments, the IgE-mediated mast cell allergic response is hay fever. In certain embodiments, the IgE-mediated mast cell allergic response is asthma. In certain embodiments, the IgE-mediated mast cell allergic response is arthritis. In certain embodiments, the IgE-mediated mast cell allergic response is urticaria. In certain embodiments, the IgE-mediated mast cell allergic response is atopic dermatitis. In certain embodiments, the IgE-mediated mast cell allergic response is rash. In certain embodiments, the IgE-mediated mast cell allergic response is a heart disease. In certain embodiments, the IgE-mediated mast cell allergic response is multiple sclerosis.

In certain embodiments, the symptoms of the IgE-mediated mast cell allergic response include, but are not limited to, dry, cracked skin, itchiness, rash, thickened skin, and inflamed skin.

In another embodiment, provided herein is a method of treating, alleviating, or preventing one or more symptoms of an inflammatory dermal condition in a subject, comprising administering to the subject in need thereof a therapeutically effective amount of an anti-RhD antibody.

In certain embodiments, the inflammatory dermal condition is atopic dermatitis, contact dermatitis, pruritus, psoriasis, or urticaria. In certain embodiments, the inflammatory dermal condition is atopic dermatitis or eczema. In certain embodiments, the inflammatory dermal condition is contact dermatitis. In certain embodiments, the inflammatory dermal condition is pruritus. In certain embodiments, the inflammatory dermal condition is psoriasis. In certain embodiments, the inflammatory dermal condition is urticaria.

In certain embodiments, the symptoms of the inflammatory dermal condition include, but are not limited to, dry, cracked skin, itchiness, rash, thickened skin, and inflamed skin.

In certain embodiments, the subject is a human. In certain embodiments, the human subject is RhD-negative. In certain embodiments, the human subject is weak RhD-positive.

In certain embodiments, the anti-RhD antibody is a human anti-RhD antibody. In certain embodiments, the anti-RhD antibody is a polyclonal human anti-RhD antibody. In certain embodiments, the anti-RhD antibody is a monoclonal human anti-RhD antibody.

In certain embodiments, the anti-RhD antibody comprises a human IgA, IgD, IgE, IgG, IgM, or a mixture thereof. In certain embodiments, the anti-RhD antibody comprises a human IgA. In certain embodiments, the anti-RhD antibody comprises a human IgA₁ or IgA₂. In certain embodiments, the anti-RhD antibody comprises a human IgA₁. In certain embodiments, the anti-RhD antibody comprises a human IgA₂. In certain embodiments, the anti-RhD antibody comprises a human IgD. In certain embodiments, the anti-RhD antibody comprises a human IgE. In certain embodiments, the anti-RhD antibody comprises a human IgG. In certain embodiments, the anti-RhD antibody comprises a human IgG₁, IgG₂, IgG₃, or IgG₄. In certain embodiments, the anti-RhD antibody comprises a human IgG₁. In certain embodiments, the anti-RhD antibody comprises a human IgG₂. In certain embodiments, the anti-RhD antibody comprises a human IgG₃. In certain embodiments, the anti-RhD antibody comprises a human IgG₄. In certain embodiments, the anti-RhD antibody comprises a human IgM.

In certain embodiments, the anti-RhD antibody is provided as a pharmaceutical composition comprising an anti-RhD antibody and a pharmaceutically acceptable excipient. In certain embodiments, the anti-RhD antibody is provided as a pharmaceutical composition comprising an anti-RhD antibody in the amount of 300 μg or 1,500 IU per dose. In certain embodiments, the anti-RhD antibody is formulated as RHOGAM®. In certain embodiments, the anti-RhD antibody is provided as a pharmaceutical composition comprising an anti-RhD antibody in the amount of 50 μg or 250 IU per dose. In certain embodiments, the anti-RhD antibody is formulated as MICRHOGAM®.

A method provided herein encompasses treating a subject regardless of subject's age, although some diseases are more common in certain age groups.

In certain embodiments, the therapeutically effective amount of the anti-RhD antibody is ranging from about 1 to about 5,000 μg per administration, from about 10 to about 2,000 μg per administration, from about 20 to about 1,000 μg per administration, or from about 50 to about 500 μg per administration. In certain embodiments, the therapeutically effective amount of the anti-RhD antibody is ranging from about 1 to about 5,000 μg per administration. In certain embodiments, the therapeutically effective amount of the anti-RhD antibody is ranging from about 10 to about 2,000 μg per administration. In certain embodiments, the therapeutically effective amount of the anti-RhD antibody is ranging from about 20 to about 1,000 μg per administration. In certain embodiments, the therapeutically effective amount of the anti-RhD antibody is ranging from about 50 to about 500 μg per administration. In certain embodiments, the therapeutically effective amount of the anti-RhD antibody is about 50, about 100, about 150, about 200, about 250, about 300, about 350, about 400, about 450, or about to about 500 μg per administration.

In certain embodiments, the anti-RhD antibody is administered once a week, once every two weeks, once every three weeks, once a month, once every two months, once every three months, once every six months, or once a year. In certain embodiments, the anti-RhD antibody is administered once a week. In certain embodiments, the anti-RhD antibody is administered once every two weeks. In certain embodiments, the anti-RhD antibody is administered once every three weeks. In certain embodiments, the anti-RhD antibody is administered once a month. In certain embodiments, the anti-RhD antibody is administered once every two months. In certain embodiments, the anti-RhD antibody is administered once every three months. In certain embodiments, the anti-RhD antibody is administered once every six months. In certain embodiments, the anti-RhD antibody is administered once a year.

In certain embodiments, the anti-RhD antibody is administered parenterally. In certain embodiments, the anti-RhD antibody is administered intravenously. In certain embodiments, the anti-RhD antibody is administered intramuscularly. In certain embodiments, the anti-RhD antibody is administered subcutaneously. In certain embodiments, the anti-RhD antibody is administered topically.

The active ingredient described herein, that is, the anti-RhD antibody, can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of the active ingredient described herein to a subject. In certain embodiments, the kit provided herein includes a container and a dosage form of the active ingredient.

Kits provided herein can further include devices that are used to administer the active ingredient. Examples of such devices include, but are not limited to, syringes, needle-less injectors drip bags, patches, and inhalers.

Kits provided herein can further include pharmaceutically acceptable vehicles that can be used to administer the active ingredient. For example, if the active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

### Example 1

### Treatment of Atopic Dermatitis with RHOGAM®

An adult female human subject with severe atopic dermatitis having the symptoms of itchiness and hives was treated once with RHOGAM® (300 μg, 1,500 IU) via intramuscular injection. Her symptoms started to disappear within 2 days and her skin was clear within 2 weeks. Some symptoms began to return after 2 months of the treatment.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating, alleviating, or preventing one or more symptoms of an inflammatory dermal condition in a subject, comprising administering to the subject in need thereof a therapeutically effective amount of an anti-RhD antibody.

2. A method of treating, alleviating, or preventing one or more symptoms of an IgE-mediated mast cell allergic response in a subject, comprising administering to the subject in need thereof a therapeutically effective amount of an anti-RhD antibody.

3. The method of claim 2, wherein the IgE-mediated mast cell allergic response is an inflammatory dermal condition.

4. The method of claim 2, wherein the IgE-mediated mast cell allergic response is psoriasis.

5. The method of claim 2, wherein the IgE-mediated mast cell allergic response is an allergy, Type I hypersensitivity, anaphylaxis, hay fever, asthma, arthritis, urticaria, atopic dermatitis, rash, a heart disease, or multiple sclerosis.

6. The method of claim 1 or 3, wherein the inflammatory dermal condition is atopic dermatitis, contact dermatitis, pruritus, psoriasis, or urticaria.

7. The method of any one of claims 1, 3, and 6, wherein the inflammatory dermal condition is atopic dermatitis.

8. The method of any one of claims 1, 3, and 6, wherein the inflammatory dermal condition is contact dermatitis.

9. The method of any one of claims 1, 3, and 6, wherein the inflammatory dermal condition is pruritus.

10. The method of any one of claims 1, 3, and 6, wherein the inflammatory dermal condition is urticaria.

11. The method of any one of claims 1 to 10, wherein the subject is RhD-negative.

12. The method of any one of claims 1 to 10, wherein the subject is weak RhD-positive.

13. The method of any one of claims 1 to 12, wherein the subject is a human.

14. The method of any one of claims 1 to 13, wherein the anti-RhD antibody is a human anti-RhD antibody.

15. The method of any one of claims 1 to 14, wherein the anti-RhD antibody is formulated as RHOGAM® or MICRHOGAM®.

16. The method of any one of claims 1 to 15, wherein the anti-RhD antibody is administered parenterally.

17. The method of any one of claims 1 to 14 and 16, wherein the anti-RhD antibody is administered intravenously.

18. The method of any one of claims 1 to 16, wherein the anti-RhD antibody is administered intramuscularly.

19. The method of any one of claims 1 to 16, wherein the anti-RhD antibody is administered subcutaneously.

20. The method of any one of claims 1 to 14, wherein the anti-RhD antibody is administered topically.

21. The method of any one of claims 1 to 20, wherein the therapeutically effective amount of the anti-RhD antibody is ranging from about 1 to about 5,000 μg per administration.

22. The method of any one of claims 1 to 21, wherein the therapeutically effective amount of the anti-RhD antibody is ranging from about 10 to about 2,000 μg per administration.

23. The method of any one of claims 1 to 22, wherein the therapeutically effective amount of the anti-RhD antibody is ranging from from about 20 to about 1,000 μg per administration.

24. The method of any one of claims 1 to 23, wherein the therapeutically effective amount of the anti-RhD antibody is ranging from about 50 to about 500 μg per administration.

25. The method of any one of claims 1 to 24, wherein the anti-RhD antibody is administered once a week, once every two weeks, once every three weeks, once a month, once every two months, once every three months, once every 6 months, or once a year.

26. The method of any one of claims 1 to 25, wherein the anti-RhD antibody is administered once a month.

27. The method of any one of claims 1 to 25, wherein the anti-RhD antibody is administered once every two months.

28. The method of any one of claims 1 to 25, wherein the anti-RhD antibody is administered once every three months.

29. The method of any one of claims 1 to 25, wherein the anti-RhD antibody is administered once every six months.
